(19) United States
(12) Patent Application Publication
(10) Pub. No.: US 2024/0325700 A1
(43) Pub. Date: Oct. 3, 2024

(54) STANDARD TRACHEAL DILATOR KIT (TDK-S) AND QUADRITUBE FOR TRACHEAL STENOSIS DILATATION

(71) Applicant: Qatar Foundation for Education, Science and Community Development, Doha (QA)

(72) Inventors: Nabil Shallik, Doha (QA); Rashid Mazhar Ul Haq Siddiqui, Doha (QA)

(21) Appl. No.: 18/614,260

(22) Filed: Mar. 22, 2024

Related U.S. Application Data
(60) Provisional application No. 63/492,408, filed on Mar. 27, 2023.

(51) Int. Cl. A61M 29/02; A61M 16/04

(52) U.S. Cl. CPC A61M 29/02; A61M 16/044; A61M 2029/025

(57) ABSTRACT

Methods, systems, and apparatus are disclosed herein for a tracheal dilator kit (TDK), comprising a set of Tracheal dilators while maintaining the patient's ventilation, comprising a quadritube Tracheal dilator (TD-Q) for very tight Tracheal stenosis (<6 mm) and a standard Tracheal dilator (TD-S) for tracheal stenosis dilatation of less tight stenosis. The standard tracheal dilator (TD-S) is a three-lumen tube which includes an endotracheal tube with a rigid dilator tip with a ventilatory lumen, an endotracheal tube (ETT) cuff, and a dilation balloon. The quadritube dilator is a four-lumen tube with a first end and second end, where the dilator tip is located on the first end. The ETT cuff is located on the first end, the dilation balloon is located on the first end.

### PRIORITY CLAIM AND CROSS-REFERENCE TO RELATED APPLICATIONS

The present application claims the benefit of U.S. Provisional Application No. 63/492,408, entitled STANDARD TRACHEAL DILATATOR KIT (TDK-S) AND QUADRITUBE FOR TRACHEAL STENOSIS DILATATION and filed Mar. 27, 2023, the contents of which are hereby incorporated by reference in their entirety.

### FIELD

The present disclosure relates generally to a tracheal dilator kit. More specifically, this disclosure relates to a tracheal dilator kit (TDK) comprising a standard tracheal dilator (TD-S) and quadritube tracheal dilator (TD-Q) for graduated dilatation of tracheal stenosis.

### BACKGROUND

The COVID-19 pandemic has massively increased the need for prolonged invasive mechanical ventilation (IMV), which has led to an increase in the incidence of significant tracheal stenosis (TS) compared to the pre-COVID times. Relatedly, endoscopic dilatation of tracheal stenosis is safer than surgery.

However, the current tools and methods for TS dilatation are heterogeneous, difficult, expensive, and require a team of experts from various fields, e.g., Thoracic surgery, ENT, Head & neck surgery, Perfusion, ICU, and anesthesia.

As such, a need exists for a tracheal dilator kit (TDK) which can dilate the whole spectrum of Tracheal Stenosis (TS), from very narrow TS to the relatively larger TS, while maintaining the ventilation of the patient.

To fulfill this, a set of TDK is presented herewith which comprise: A) Quadritube Tracheal dilator (TD-Q) which is used for TS<6 mm diameter—for use with specialized ventilators (flow controlled ventilators), which allows ventilation through small airway lumens; and B) standard tracheal dilator (TD-S) which is used for TS>6 mm diameter—for use with ordinary ventilators. These two embodiments make a continuum as a patient with very narrow TS would be treated first with the TD-Q and then graduated to dilatation with TD-S. Hence both of these embodiments may be used in combination or individually.

### SUMMARY

Example systems, methods, and apparatus are disclosed herein for a tracheal dilator kit (TDK) for tracheal stenosis dilatation.

In light of the disclosure herein, and without limiting the scope of the invention in any way, in a first aspect of the present disclosure, which may be combined with any other aspect listed herein unless specified otherwise, a tracheal dilator kit (TDK) including a standard tracheal dilator (TD-S), a quadritube tracheal dilator (TD-Q), a dilator tip, an endotracheal tube (ETT) cuff, and a dilation balloon. The quadritube tracheal dilator (TD-Q) is a four-lumen tube with a first end and second end, the dilator tip located on the first end, the ETT cuff is located on the first end, and the dilation balloon is located on the first end. The standard tracheal dilator (TD-S) is a three-lumen tube with a first end and second end, the dilator tip located on the first end, the ETT cuff is located on the first end, and the dilation balloon is located on the first end.

In a second aspect of the present disclosure, which may be combined with any other aspect listed herein unless specified otherwise, the dilator tip is located at the end of the first end.

In a third aspect of the present disclosure, which may be combined with any other aspect listed herein unless specified otherwise, the ETT cuff is located a first distance away from the dilator tip.

In a fourth aspect of the present disclosure, which may be combined with any other aspect listed herein unless specified otherwise, the dilation balloon is located a second distance away from the dilator tip.

In a fifth aspect of the present disclosure, which may be combined with any other aspect listed herein unless specified otherwise, the first distance is shorter than the second distance.

In a sixth aspect of the present disclosure, which may be combined with any other aspect listed herein unless specified otherwise, further including an ETT cuff inflator tube, a pressure measurement tube, a balloon inflator tube, and a ventilation lumen. The ETT cuff inflator tube, pressure measurement tube, balloon inflator tube, and ventilation lumen each separately couple to the quadritube tracheal dilator (TD-Q) at the second end.

In a seventh aspect of the present disclosure, which may be combined with any other aspect listed herein unless specified otherwise, each of the ETT cuff inflator tube, pressure measurement tube, balloon inflator tube, and ventilation lumen each couple to one of the four lumens in the four-lumen quadritube tracheal dilator (TD-Q).

In an eighth aspect of the present disclosure, which may be combined with any other aspect listed herein unless specified otherwise, further including an ETT cuff inflation syringe. The ETT cuff inflation syringe couples to the ETT cuff inflator tube, and the ETT cuff inflator tube directs air from the ETT cuff inflation syringe to the ETT cuff to fill the ETT cuff.

In a ninth aspect of the present disclosure, which may be combined with any other aspect listed herein unless specified otherwise, further including a dilation balloon syringe. The dilation balloon inflation syringe couples to the balloon inflator tube, and the balloon inflator tube directs air from the dilation balloon inflation syringe to the dilation balloon to fill the dilation balloon.

In a tenth aspect of the present disclosure, which may be combined with any other aspect listed herein unless specified otherwise, further including a guide wire. The guide wire couples to the balloon inflator tube, and the guide wire moves from the balloon inflator tube to the first end.

In an eleventh aspect of the present disclosure, which may be combined with any other aspect listed herein unless specified otherwise, further including an ETT stylet. The ETT stylet is configured to be introduced into the ventilation lumen, the ETT stylet is selectively deformable, and the quadritube tracheal dilator (TD-Q) is configured to selectively deform to conform to the shape of the ETT stylet.

In a twelfth aspect of the present disclosure, which may be combined with any other aspect listed herein unless specified otherwise, the ventilation lumen is configured to selectively couple with a ventilator.

In a thirteenth aspect of the present disclosure, which may be combined with any other aspect listed herein unless specified otherwise, further including a connector with at least two connection ports. The connector couples to the balloon inflator tube.

In a fourteenth aspect of the present disclosure, which may be combined with any other aspect listed herein unless specified otherwise, further including a pressure-measuring component with pressure sensors. The pressure-measuring component couples to the pressure measurement tube, and the pressure measurement tube directs the pressure sensors to the first end to measure pressure in the first end.

In a fifteenth aspect of the present disclosure, which may be combined with any other aspect listed herein unless specified otherwise, a tracheal dilating system including a ventilator, a pressure-measurement device with pressure sensors, a standard tracheal dilator kit. The standard tracheal dilator (TDK-S) kit including a dilator tip, an endotracheal tube (ETT) cuff, a dilation balloon, a standard tracheal dilator (TD-S), and a quadritube tracheal dilator (TD-Q). The quadritube tracheal dilator (TD-Q) is a four-lumen tube with a first end and second end, the dilator tip located on the first end, the ETT cuff is located on the first end, and the dilation balloon is located on the first end.

In a sixteenth aspect of the present disclosure, which may be combined with any other aspect listed herein unless specified otherwise, the standard tracheal dilator kit (TDK-S) further comprises an ETT cuff inflator tube, a pressure measurement tube, a balloon inflator tube, and a ventilation lumen, wherein the ETT cuff inflator tube, pressure measurement tube, balloon inflator tube, and ventilation lumen each separately couple to the quadritube tracheal dilator (TD-Q) at the second end.

In a seventeenth aspect of the present disclosure, which may be combined with any other aspect listed herein unless specified otherwise, each of the ETT cuff inflator tube, pressure measurement tube, balloon inflator tube, and ventilation lumen each couple to one of the four lumens in the four-lumen quadritube.

In an eighteenth aspect of the present disclosure, which may be combined with any other aspect listed herein unless specified otherwise, the pressure-measurement device couples to the pressure measurement tube, and the pressure measurement tube directs the pressure sensors to the first end to measure pressure in the first end.

In a nineteenth aspect of the present disclosure, which may be combined with any other aspect listed herein unless specified otherwise, the standard tracheal dilator kit (TDK-S) further comprises a dilation balloon syringe, a guide wire, and an ETT stylet. The dilation balloon inflation syringe couples to the balloon inflator tube. The balloon inflator tube directs air from the dilation balloon inflation syringe to the dilation balloon to fill the dilation balloon. The guide wire couples to the balloon inflator tube. The guide wire moves from the balloon inflator tube to the first end. The ETT stylet is configured to be introduced into the ventilation lumen. The ETT stylet is selectively deformable. The quadritube tracheal dilator (TD-Q) is configured to selectively deform to conform to the shape of the ETT stylet.

In a twentieth aspect of the present disclosure, which may be combined with any other aspect listed herein unless specified otherwise, a quadritube configured for use in a tracheal dilator kit (TDK). The quadritube is a four-lumen tube with a first end and second end, where the first end includes a dilator tip, and each of the four lumens at the second end is configured to receive and couple to one of an ETT cuff inflator tube, a pressure measurement tube, a balloon inflator tube, and a ventilation lumen.

In a twenty-first aspect of the present disclosure, which may be combined with any other aspect listed herein unless specified otherwise, a standard tracheal dilator (TD-S) is configured for use in a tracheal dilator kit (TDK) in combination with the quadritube (TD-Q). The standard tracheal dilator (TD-S) is a three-lumen tube with a first end and second end, where the first end includes a rigid dilator tip on the first end, the ETT cuff is located on the first end, and the dilation balloon is located on the first end. Each of the three lumens at the second end is configured to receive and couple to one of an ETT cuff inflator tube, a pressure measurement tube, a balloon inflator tube, and a ventilation lumen.

In a twenty-second aspect of the present disclosure, any of the structure, functionality, and alternatives disclosed in connection with any one or more of FIGS. 1 to 5 may be combined with any other structure, functionality, and alternatives disclosed in connection with any other one or more of FIGS. 1 to 5.

In light of the present disclosure and the above aspects, it is therefore an advantage of the present disclosure to provide users with a dilator kit comprising standard tracheal dilator (TD-S) and quadritube (TD-Q) for tracheal stenosis dilatation.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. In addition, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a diagram view of a quadritube tracheal dilator (TD-Q), according to an example embodiment of the present disclosure.

FIG. 2 is a top view of a quadritube tracheal dilator kit (TD-Q), according to an example embodiment of the present disclosure.

FIG. 3 is a top view of a quadritube tracheal dilator kit (TD-Q), according to an example embodiment of the present disclosure.

FIG. 4 is a flow diagram of a method of using a standard tracheal dilator (TD-S), of the tracheal dilator kit (TDK), according to an example embodiment of the present disclosure.

FIG. 5 is a diagram view of a standard tracheal dilator (TD-S), according to an example embodiment of the present disclosure.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specific the presence of stated features, integers, steps, operations, elements, and/or components, but do not preclude the presence or additional of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent”). As used herein, the term “and/or”includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Methods, systems, and apparatus are disclosed herein for a tracheal dilator kit (TDK) comprising a standard tracheal dilator kit (TDK-S) and quadritube (TD-Q) for tracheal stenosis dilatation.

While the example methods, apparatus, and systems are disclosed herein a standard tracheal dilator kit (TDK-S) and quadritube for tracheal stenosis dilatation, it should be appreciated that the methods, apparatus, and systems may be operable for other medical dilatations.

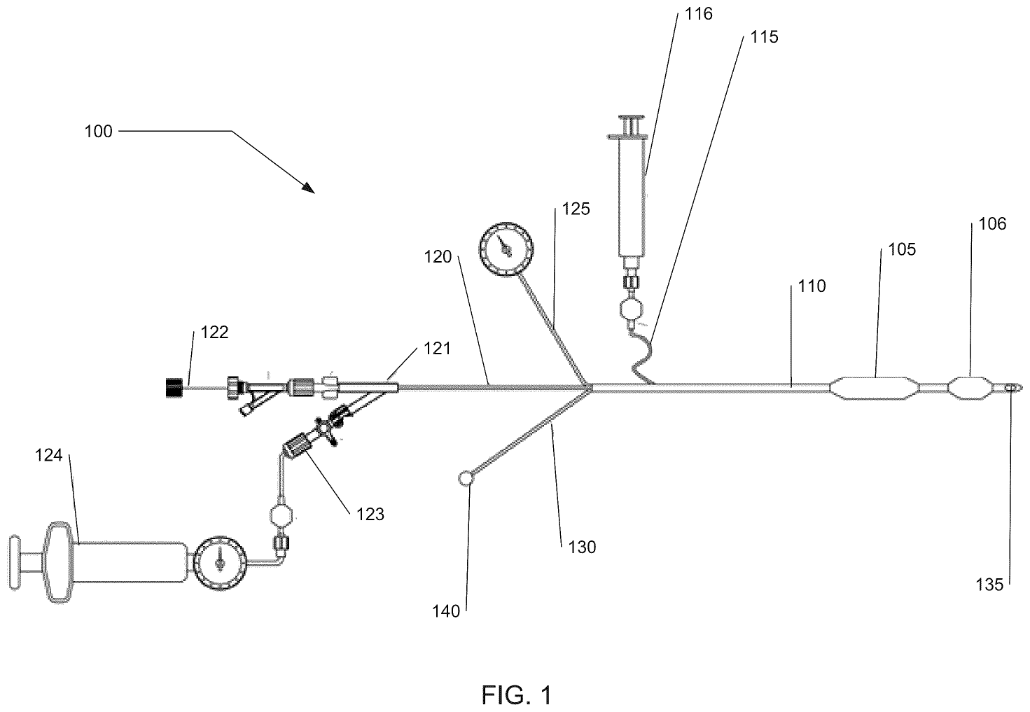

FIG. 1 is a diagram of a quadritube 100. As seen in FIG. 1, the quadritube 100 includes a dilator tip 135, an endotracheal tube (ETT) cuff 106, a dilation balloon 105, and a quadritube endotracheal tube 110. Four different sources feed into the quadritube's endotracheal tube 110. As such, the quadritube endotracheal tube 110 is a 4-lumen tube, where each lumen corresponds with a different of the four sources. The four sources are an ETT cuff inflator tube 115, a pressure measurement tube 125, a balloon inflator tube 120, and a ventilation lumen 130. The quadritube endotracheal tube 110 can be made of medical-grade plastic, or any other medically safe pliable material, like silicone.

As also seen in FIG. 1, the ETT cuff inflator tube 115 is selectively coupled to an ETT cuff inflation syringe 116. The pressure measurement tube 125 is selectively coupled to a pressure gage or other pressure-monitoring device. The ventilation lumen 130 includes an ETT stylet 140, and the ventilation lumen 130 connects to a ventilator (not shown). The ballon inflator tube 120 includes a connector 121, such as a T-connector, with at least two entry ports. A guide wire 122 is fed through one of the entry ports of the connector 121, and a dilation balloon inflator 123 connects to the other entry port. The dilation balloon inflator 123 connects to a balloon inflation syringe 124. While the preferred embodiment includes an ETT cuff inflation syringe 116 and a balloon inflation syringe 124, alternate embodiments may include different components to inflate the ETT cuff 106 and dilation balloon 105.

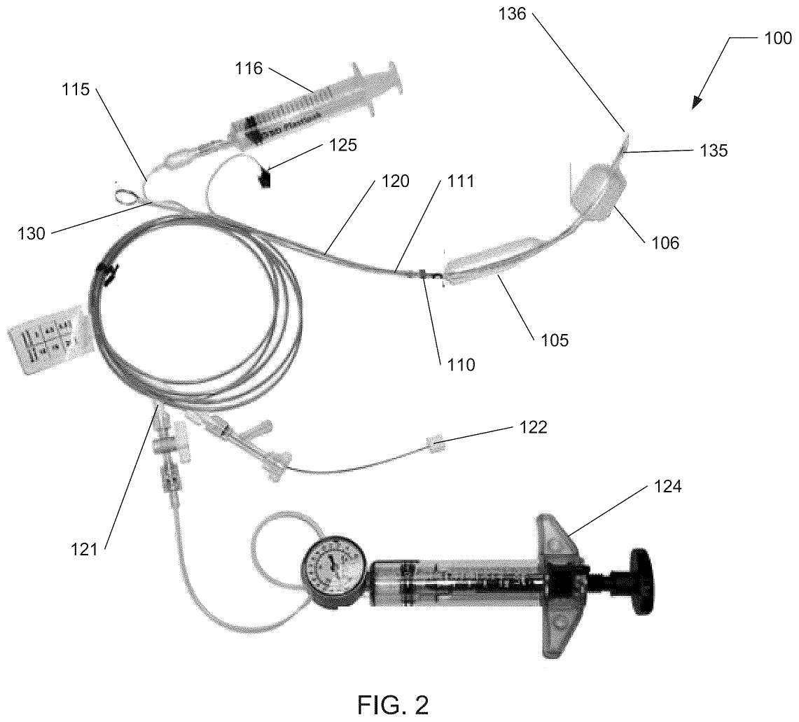

Relatedly, FIG. 2 shows a top view of the quadritube 100. As seen in FIG. 2, the quadritube 100 includes a patient end 136 and a quadritube end 111. The patient end 136 is inserted into the patient during a tracheal stenosis and the quadritube end 111, opposite the patient end 136, is the entrance point for the ETT cuff inflator tube 115, pressure measurement tube 125, balloon inflator tube 120, and ventilation lumen 130.

During use, the ETT cuff inflator tube 115 directs air from the ETT cuff inflation syringe 116, through the quadritube endotracheal tube 110, and to the ETT cuff 106, to inflate the ETT cuff 106. Similarly, during use, the pressure measurement tube 125 directs sensors from the quadritube end 111 to the patient end 136, to measure the pressure on the patient's trachea. The balloon inflator tube 120 directs both the guide wire 122 and air from the balloon inflation syringe 124 through the quadritube endotracheal tube 110 and to the patients end 136, where the guide wire 122 guides the dilation balloon 105 and the air inflates the dilation balloon 105. Finally, the ventilation lumen 130 directs the ETT stylet 140 through the quadritube endotracheal tube 110 and to the patient end 136. The user can manipulate the shape of the ETT stylet 140 such that the quadritube endotracheal tube 110 is directed accordingly down the patient's trachea. Next, the user can connect a ventilator, or other assistive device, to the ventilation lumen 130.

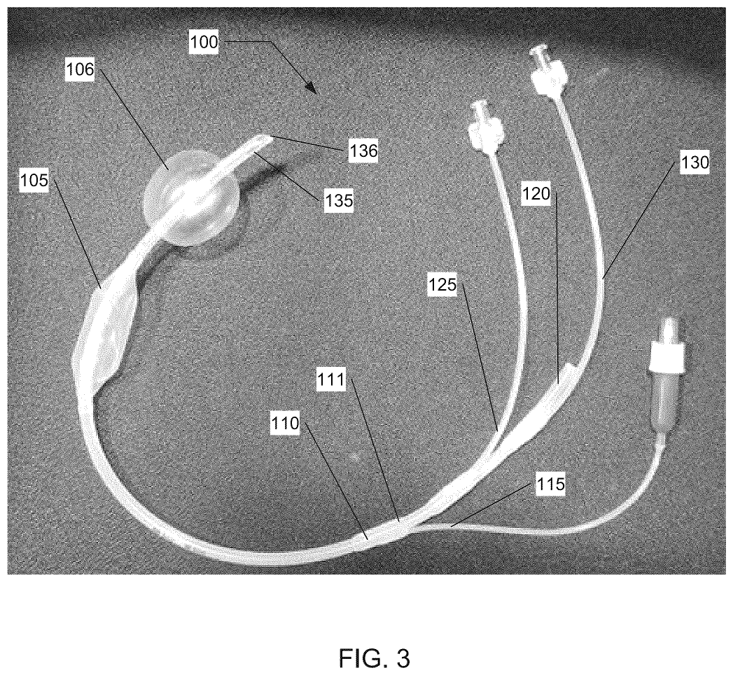

FIG. 3 shows a top view of the quadritube 100. As previously described, quadritube 100 includes a dilator tip 135, an ETT cuff 106, a balloon 105, and a quadritube endotracheal tube 110 into which an ETT cuff inflator tube 115, a pressure measurement tube 125, a balloon inflator tube 120, and a ventilation lumen 130 feed. Moreover, the dilator tip 135 directs the end of the ventilation lumen 130 to the patient end 136, so as to direct air from a ventilator through a patient's trachea.

As shown in FIG. 3, the order for the components, from quadritube end 111 to patient end 136, is quadritube end 111 to quadritube 110, to dilation balloon 105, to ETT cuff 106, to dilation tip 136, and to patient end 136. In the preferred embodiment, the ETT cuff 106 is 2 centimeters from the dilator tip 135, the ETT cuff 106 is 3 centimeters from start to end, and there is a distance of 2 centimeters between the ETT cuff 106 and the dilation balloon 105. The length of the quadritube varies.

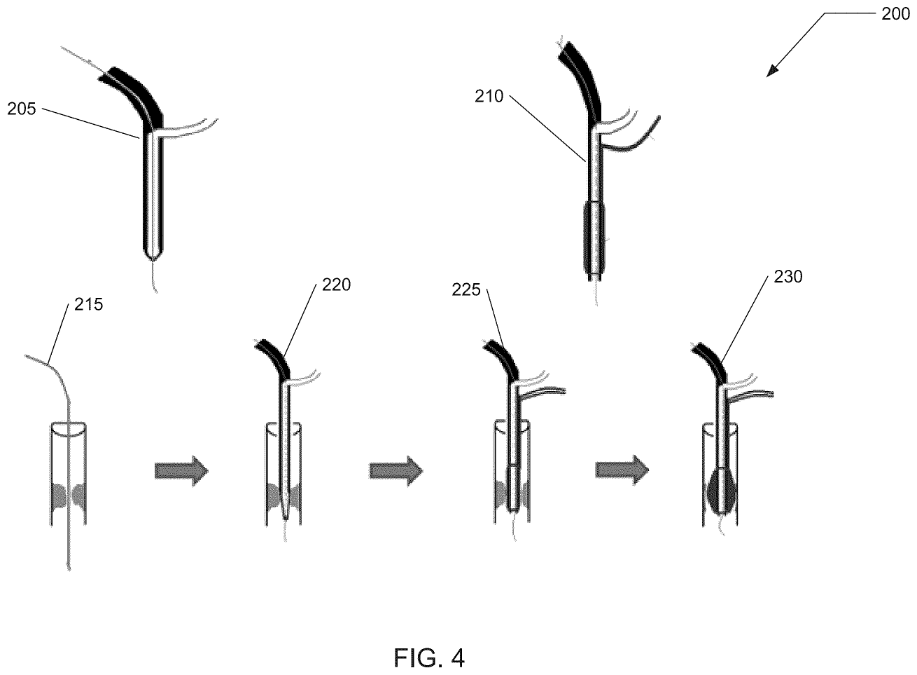

FIG. 4 is a flow diagram of a method of using a standard tracheal dilator (TD-S) 200. As seen in FIG. 4, the method 200 works based on the three step Seldinger method. As such, there is an initial state 205 where a dilator tip is inserted into a patient tracheal stenosis, dilating it by the mechanical action of its tapered end, reaching a final state 210 where a dilator balloon is inserted into the partially dilated trachea and inflated. The initial step of the process 215 involves crossing a guide wire through the tracheal stenosis. After this, a TDK-S dilator made of a firm plastic polymer is passed over the guide wire. The tapered end of the TDK-S dilator dilates the tracheal stenosis to up to about 10 mm in width. Once this happens, a balloon is fed through the dilator 225. Finally, the balloon is inflated to achieve the target dilation 230.

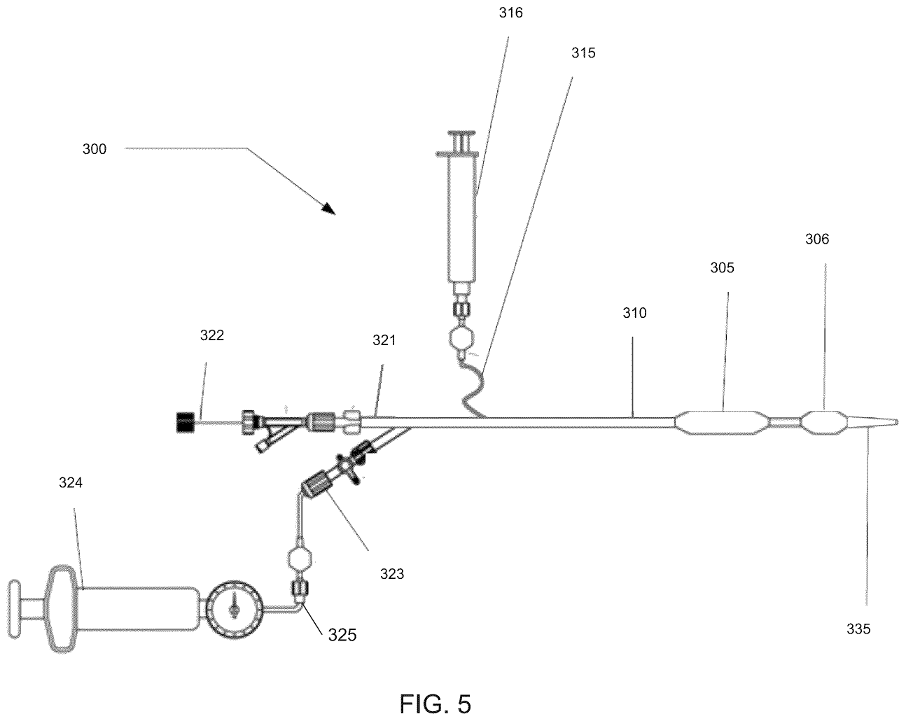

Finally, FIG. 5 is a diagram of a standard tracheal dilator (TD-S) 300. As seen in FIG. 5, the dilator kit (TD-S) 300 includes a dilator tip 335, an endotracheal tube (ETT) cuff 306, a dilation balloon 305, and a TD-S endotracheal tube 310. Three different sources feed into the TD-S endotracheal tube 310. As such, the TD-S endotracheal tube 310 is a 3-lumen tube, where each lumen corresponds with a different of the three sources. The three sources are an ETT cuff inflator tube 315, a ventilation lumen 323 and a balloon inflator tube incorporated along the ventilation lumen 323. The TD-S endotracheal tube 310 can be made of medical-grade plastic, or any other medically safe pliable material, like silicone.

As also seen in FIG. 5, the ETT cuff inflator tube 315 is selectively coupled to an ETT cuff inflation syringe 316. The ventilation lumen 323 connects to a ventilator (not shown). A guide wire 322 is fed through one of the entry ports of the connector 321, and a dilation balloon inflator 325 connects to the ventilation lumen 323. The dilation balloon inflator 325 connects to a balloon inflation syringe 324. While the preferred embodiment includes an ETT cuff inflation syringe 316 and a balloon inflation syringe 324, alternate embodiments may include different components to inflate the ETT cuff 306 and dilation balloon 305.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

The invention is claimed as follows:

1. A tracheal dilator kit (TDK), comprising:
a quadritube tracheal dilator (TD-Q),
a standard tracheal dilator (TD-S),
a dilator tip,
an endotracheal tube (ETT) cuff, and
a dilation balloon,
wherein the quadritube tracheal dilator (TD-Q) is a four-lumen tube with a first end and second end, wherein the dilator tip located on the first end, wherein the ETT cuff is located on the first end, and wherein the dilation balloon is located on the first end, and
wherein the standard tracheal dilator (TD-S) is a triple-lumen tube with a first end and second end, wherein the rigid dilator tip located on the first end, wherein the ETT cuff is located on the first end, and wherein the dilation balloon is located on the first end.

2. The tracheal dilator kit (TDK) of claim 1, wherein the dilator tip is located at the end of the first end.

3. The tracheal dilator kit (TDK) of claim 2, wherein the ETT cuff is located a first distance away from the dilator tip.

4. The tracheal dilator kit (TDK) of claim 3, wherein the dilation balloon is located a second distance away from the dilator tip.

5. The tracheal dilator kit (TDK) of claim 4, wherein the first distance is shorter than the second distance.

6. The tracheal dilator kit (TDK) of claim 1, further comprising an ETT cuff inflator tube, a pressure measurement tube, a balloon inflator tube, and a ventilation lumen, wherein the ETT cuff inflator tube, pressure measurement tube, balloon inflator tube, and ventilation lumen each separately couple to the quadritube tracheal dilator (TD-Q) at the second end.

7. The tracheal dilator kit (TDK) of claim 6, wherein each of the ETT cuff inflator tube, pressure measurement tube, balloon inflator tube, and ventilation lumen each couple to one of the four lumens in the four-lumen quadritube tracheal dilator (TD-Q).

8. The tracheal dilator kit (TDK) of claim 6, further comprising an ETT cuff inflation syringe, wherein the ETT cuff inflation syringe couples to the ETT cuff inflator tube, and wherein the ETT cuff inflator tube directs air from the ETT cuff inflation syringe to the ETT cuff to fill the ETT cuff.

9. The tracheal dilator kit (TDK) of claim 6, further comprising a dilation balloon syringe, wherein the dilation balloon inflation syringe couples to the balloon inflator tube, and wherein the balloon inflator tube directs air from the dilation balloon inflation syringe to the dilation balloon to fill the dilation balloon.

10. The tracheal dilator kit (TDK) of claim 6, further comprising a guide wire, wherein the guide wire couples to the balloon inflator tube, and wherein the guide wire moves from the balloon inflator tube to the first end.

11. The tracheal dilator kit (TDK) of claim 6, further comprising an ETT stylet, wherein the ETT stylet is configured to be introduced into the ventilation lumen, wherein the ETT stylet is selectively deformable, and wherein the quadritube tracheal dilator (TD-Q) is configured to selectively deform to conform to the shape of the ETT stylet.

12. The tracheal dilator kit (TDK) of claim 11, wherein the ventilation lumen is configured to selectively couple with a ventilator.

13. The tracheal dilator kit (TDK) of claim 6, further comprising a connector with at least two connection ports, wherein the connector couples to the balloon inflator tube.

14. The tracheal dilator kit (TDK) of claim 6, further comprising a pressure-measuring component with pressure sensors, wherein the pressure-measuring component couples to the pressure measurement tube, and wherein the pressure measurement tube directs the pressure sensors to the first end to measure pressure in the first end.

15. A tracheal dilating system, comprising:
a ventilator;
a pressure-measurement device with pressure sensors; and
a standard tracheal dilator kit (TDK-S), comprising:
a dilator tip,
an endotracheal tube (ETT) cuff,
a dilation balloon,
a standard tracheal dilator (TD-S), and
a quadritube tracheal dilator (TD-Q),
wherein the quadritube tracheal dilator (TD-Q) is a four-lumen tube with a first end and second end, wherein the dilator tip located on the first end, wherein the ETT cuff is located on the first end, and wherein the dilation balloon is located on the first end.

16. The tracheal dilating system of claim 15, wherein the standard tracheal dilator kit (TDK-S) further comprises an ETT cuff inflator tube, a pressure measurement tube, a balloon inflator tube, and a ventilation lumen, wherein the ETT cuff inflator tube, pressure measurement tube, balloon inflator tube, and ventilation lumen each separately couple to the quadritube tracheal dilator (TD-Q) at the second end.

17. The tracheal dilating system of claim 16, wherein each of the ETT cuff inflator tube, pressure measurement tube, balloon inflator tube, and ventilation lumen each couple to one of the four lumens in the four-lumen quadritube tracheal dilator (TD-Q).

18. The tracheal dilating system of claim 16, wherein the pressure-measurement device couples to the pressure measurement tube, and wherein the pressure measurement tube directs the pressure sensors to the first end to measure pressure in the first end.

19. The tracheal dilating system of claim 16, wherein the standard tracheal dilator kit (TDK-S) further comprises a dilation balloon syringe, a guide wire, and an ETT stylet, wherein the dilation balloon inflation syringe couples to the balloon inflator tube, wherein the balloon inflator tube directs air from the dilation balloon inflation syringe to the dilation balloon to fill the dilation balloon, wherein the guide wire couples to the balloon inflator tube, wherein the guide wire moves from the balloon inflator tube to the first end, wherein the ETT stylet is configured to be introduced into the ventilation lumen, wherein the ETT stylet is selectively deformable, and wherein the quadritube tracheal dilator (TD-Q) is configured to selectively deform to conform to the shape of the ETT stylet.

20. A quadritube configured for use in a tracheal dilator kit (TDK),
wherein the quadritube is a four-lumen tube with a first end and second end,
wherein the first end includes a dilator tip, and
wherein each of the four lumens at the second end is configured to receive and couple to one of an ETT cuff inflator tube, a pressure measurement tube, a balloon inflator tube, and a ventilation lumen.
